(19) United States
(12) Patent Application Publication
(10) Pub. No.: US 2011/0142857 A1
(43) Pub. Date: Jun. 16, 2011

(54) METHODS OF ALTERING PERIPHERAL B CELL POPULATIONS AND USES THEREOF

(75) Inventors: Doron Melamed, Caesarea (IL); Zohar Keren, Gesher Haziv (IL)

(73) Assignee: RAPPAPORT FAMILY INSTITUE FOR RESEACH IN THE MADICAL SCIENCES EFRON STREET, HAIFA (IL)

(21) Appl. No.: 13/054,941

(22) PCT Filed: Jul. 19, 2009
(86) PCT No.: PCT/IL09/00706

§ 371 Date: Jan. 20, 2011

(30) Foreign Application Priority Data
Jul. 23, 2008 (US) 61/129842

(51) Int. Cl. A61K 39/395; A61P 31/00; A61P 43/00

(52) U.S. Cl. 424/172.1; 424/130.1

(57) ABSTRACT

A method of altering peripheral B cell populations in a subject in need thereof is disclosed. The method comprising administering to the subject a therapeutically effective amount of an agent capable of depleting peripheral B cells in the subject, and wherein the subject does not have a hematologic cancer or an autoimmune disease, thereby altering the peripheral B cell populations in the subject.

### FIELD AND BACKGROUND OF THE INVENTION

The present invention, in some embodiments thereof, relates to methods of altering peripheral B cell populations and, more particularly, but not exclusively, to the use of same for improving immune competence.

Ageing is a complex process that negatively impacts the development of the immune system and its ability to function. It is also considered the most common immune deficiency state and immune dysregulation. As a result, the elderly population suffers from a heightened susceptibility to infectious diseases and the major cause of morbidity and mortality among the elderly is from a dramatic decline in the immune system's ability to mount protective responses. The aging of the immune system involves many physiological changes that are collectively referred to as “immune senescence”. These changes affect both the innate and adaptive immune systems. The most important immunological manifestations in aging include poor responsiveness to new or evolving pathogens and reduced efficacy to vaccination. The primary causes of this immune incompetence are the decline in production of naïve lymphocytes in the bone marrow (BM) and thymus and the expansion and increased survival of antigen-experienced memory lymphocytes. The consequential outcome of these changes is a marked reduction in the diversity of the peripheral lymphocyte repertoire and in the capacity of the body to mount protective antibody responses [Weng, Immunity (2006) 24, 495-499; Linton and Dorshkind, Nat. Immunol. (2004) 5, 133-139].

The B lineage undergoes dramatic age-related alterations in its cellular composition. Old mice show a marked decrease in the frequency of precursor B cells and B-cell production in the BM, whereas in the periphery there is a significant reduction in naïve follicular B cells and accumulation of long-lived antigen-experienced B cells [Johnson et al., J. Immunol. (2002) 168, 5014-5023; Miller and Allman, J. Immunol. (2003) 171, 2326-2330]. Thus, with aging, the antibody response to new antigenic insult is poor both in quality and quantity, since it depends on a less diverse repertoire that is dominated by the antigen-experienced B cells [Weksler, Vaccine (2000) 18, 1624-1628].

The mechanisms underlying these cellular alterations are unclear. Most existing data support the idea that hematopoietic stem cells (HSCs) acquire intrinsic defects (changes in transcription regulatory proteins and responsiveness to growth factors) with aging that suppress B lymphopoiesis [Guerrettaz et al., Proc Natl Acad Sci USA (2008) 105, 11898-11902; Rossi et al. Proc Natl Acad Sci USA (2005) 102, 9194-9199]. Other studies have shown that senescence of B lymphopoiesis in the BM results from B cell extrinsic (reduced production of growth factors, competition for growth niches) factors that are stage specific and developmentally regulated [Tsuboi et al., Exp Biol Med (Maywood) (2004) 229:494-502; Miller and Allman, J Immunol (2003) 171:2326-2330; Frasca et al., J Immunol (2008) 180:2741-2746]. Either way, this process is regarded as irreversible [Li et al., Eur. J. Immunol. (2001) 31, 500-505]. Accordingly, the peripheral B-cell compartment adapts to these changes by accumulating long-lived cells, thus maintaining the peripheral B-cell numbers unchanged.

Currently, no treatment or drug is available to overcome this physiological obstacle. Most attempts to enhance efficacy of vaccination in the elderly are based on improving the delivery methods, however, these have yielded limited success [Zheng et al., Clin. Immunol. (2007) 124, 131-137]. Some attempts have also been made to promote B cell lymphopoiesis, these are summarized infra.

U.S. Pat. No. 7,504,105 discloses a novel human polypeptide named Cytokine Receptor Common Gamma Chain Like (CRCGCL) which may be used to promote B cell lymphopoiesis, for example, to boost immune response and/or recovery in the elderly and immunocompromised individuals.

U.S. Pat. No. 5,554,595 discloses methods of enhancing B cell lymphopoiesis in the bone marrow using selective hormones. Accordingly, U.S. Pat. No. 5,554,595 teaches the use of hormones, such as estrogens, estrogen-like compounds, and related steroids, for influencing B lymphopoiesis and treating disorders (e.g. autoimmune disorders).

Furthermore, several drugs, which are based on B cell depletion and/or dilution, have been approved for treatment in humans. For example, the anti-CD20 monoclonal antibody (e.g. Rituximab) is considered as a very safe drug that is very-well tolerated in most patients with minimal complications. Anti-CD20 has been used for the treatment of autoimmunity disorders, such as Rheumatoid Arthritis [Edwards and Cambridge, Nat Rev Immunol. (2006) 6: 394-403], lupus [Tahir et al., Rheumatology (Oxford) (2005) 44(4):561-2. Epub 2005 Jan. 11] and autoimmune thrombocytopenia in chronic graft-versus-host disease [Ratanatharathorn et al., Annals of Inter Med (2000) 133(4):275-279], for the treatment of B-cell non-Hodgkin's lymphoma (NHL) and chronic lymphocytic leukemia (CLL) [Milani and Castillo, Curr Opin Mol Ther. (2009) 11(2):200-7]. Also, the anti-Blys (BAFF) monoclonal antibody (e.g. Belimumab) has entered clinical trials for treatment of autoimmune disorders such as lupus and rheumatoid arthritis [Drugs R D. (2008) 9(3):197-202].

### SUMMARY OF THE INVENTION

According to an aspect of some embodiments of the present invention there is provided a method of altering peripheral B cell populations in a subject in need thereof, the method comprising administering to the subject a therapeutically effective amount of an agent capable of depleting peripheral B cells in the subject, and wherein the subject does not have a hematologic cancer or an autoimmune disease, thereby altering the peripheral B cell populations in the subject. According to an aspect of some embodiments of the present invention there is provided a use of an agent capable of depleting peripheral B cells for the manufacture of a medicament identified for treating a medical condition selected from the group consisting of an immune deficiency, a solid tumor, an inflammatory disease, an infectious disease and a transplantation-related disease, and further wherein the medical condition is not a hematologic cancer or an autoimmune disease.

According to an aspect of some embodiments of the present invention there is provided a use of an agent capable of depleting peripheral B cells for treating a medical condition selected from the group consisting of an immune deficiency, a solid tumor, an inflammatory disease, an infectious disease and a transplantation-related disease, and further wherein the medical condition is not a hematologic cancer or an autoimmune disease.

According to some embodiments of the invention, the therapeutically effective amount is sufficient to allow generation of new B cells in the bone marrow of the subject.

According to some embodiments of the invention, the subject is a human subject.

According to some embodiments of the invention, the subject is immune compromised.

According to some embodiments of the invention, the subject is age-related immune compromised.

According to some embodiments of the invention, the subject is at least about 40 years old.

According to some embodiments of the invention, the subject has a medical condition selected from the group consisting of an immune deficiency, a solid tumor, an inflammatory disease, an infectious disease and a transplantation-related disease.

According to some embodiments of the invention, the agent comprises a targeting moiety.

According to some embodiments of the invention, the targeting moiety comprises an antibody.

According to some embodiments of the invention, the antibody comprises an anti-B cell antibody.

According to some embodiments of the invention, the anti-B cell antibody is selected from the group consisting of an anti-CD20 antibody, an anti-CD22 antibody and an anti-CD19 antibody.

According to some embodiments of the invention, the antibody comprises an antibody targeting a B cell survival factor.

According to some embodiments of the invention, the antibody targeting a B cell survival factor comprises an anti-Blys (BAFF) antibody.

According to some embodiments of the invention, the administering is effected for a chronic treatment.

According to some embodiments of the invention, the administering is effected for an acute treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-R are dot plot graphs depicting that B lineage in old CD19−/− and Ii−/− mice does not become senescent. The B lineage of old (more than 20 months) and young (3-4 months) B10D2 wild type (wt), B10D2 CD19−/−, or C57B16 Ii−/− mice was analyzed. FIGS. 1A-L show bone marrow (BM) cells which were stained for B220, IgM and AA4.1 surface markers and analyzed by flow cytometry. FIGS. 1A-F show analysis of IgM and B220 and distinguished between pro/pre (bottom panel), immature (upper left panel), and circulating B cells (upper right panel). FIGS. 1G-L show analysis of IgM and AA4.1 and was conducted on gated B220+ cells. FIGS. 1M-R show spleen cells which were stained for IgM, B220 and PanCD45. Analysis for B220 and PanCD45 was conducted on gated IgM+ cells. Plots shown are representative of 4-5 mice in each group.

FIG. 1S is a bar graph depicting a maintained high output of B lymphopoiesis in old CD19−/− mice. To measure the output capacity of BM, inventors performed time-course experiments. Young and old B10D2 wt or CD19−/− mice were subjected to B-cell depletion by a mixture of monoclonal antibodies. To determine the kinetics of B-cell return, peripheral blood cells were collected before and up to 50 days after the depletion and analyzed for B220 and AA4.1 expression (by FACS). The percentage of immature (AA4.1+) and mature (AA4.1−) B cells over time is shown. Results are the mean ±SE of at least 5 mice in each group.

FIGS. 2A-G are graphs depicting antibody-mediated B-cell depletion and kinetics of B-cell return. A mixture of monoclonal antibodies was injected i.p. into five B10D2 young (3-4 months) wt mice. The mice were bled before and at different time points after injection of the antibodies. The cells were stained for CD19, NK1.1, and CD5 to estimate the B-cell depletion efficiency and to follow the return of the B cells. FIGS. 2A-B are dot plot graphs showing flow-cytometry results of B-cell numbers in blood samples of young mice before depletion. FIGS. 2C-D are dot plot graphs showing flow-cytometry results of B-cells depletion in blood samples collected 3 days after the last injection in young mice. FIG. 2E is a line graph showing the kinetics of B-cell return as reflected in the % B cells in the peripheral blood as measured by flow cytometry. FIG. 2F is a bar graph depicting B-cell depletion in the indicated organs. Single-cell suspensions from the indicated organs were prepared on day 3 after the last antibody injection, cells were stained for B220, CD19, and IgM and were analyzed by FACS (n=3). The B-cell numbers were determined from the total cell counts and the relative frequencies shown by FACS (total counts for B cells in the control untreated mice were: 60±12% in peripheral blood, 5±0.7×10⁵ in peritoneal cavity, 2.5±0.9×10⁶ in mesenteric lymph node 26±12×10⁶ in the spleen and 22±7×10⁶ in the BM). The % B-cell depletion was determined as the number of B cells in the respective organ in the treated mice divided by the number of B cells found in same organ in the control aged-matched wt mice and multiplied by 100. FIG. 2G is a bar graph depicting the total number of B cells in the spleen. Mice were sacrificed at different time intervals (n=3 at each time point). Spleen cells were counted and stained to determine percentages of B220+ B cells. The total number of B cells was determined by multiplying the number of cells in the spleen by the % B cells as measured by FACS.

FIGS. 3A-F are dot plot graphs depicting a maintained young-like peripheral B-cell repertoire in old CD19-deficient mice. To study repertoire changes, inventors used the 3-83 Tg mouse model. Splenic cells from young and old B10D2 3-83 Tg and B10D2 3-83 Tg CD19−/− mice were stained for B220, IgM, PanCD45, and the anti-3-83 idiotypic Ab (54.1) and then analyzed by flow cytometry. FIGS. 3A-D show lymphocytes which were gated for B220+ and analyzed for IgM vs. anti-3-83 idiotype. FIGS. 3E-F show lymphocytes which were gated for IgM+ and analyzed for B220 vs. PanCD45. The plots represent 5 mice in each group.

FIGS. 4A-G are dot plot graphs depicting a reduced number of B cells in the blood and an increased fraction of young AA4.1+ B cells following ablation of BAFF-R in vivo. FIGS. 4A-D, young mice (n=3) which were C57B16 BAFF-R^{FL/FL}/MX-cre or control C57B16 BAFF-R^{FL/+}/MX-cre were injected with poly(I)-poly(C) to ablate the floxed-BAFF-R allele. Peripheral blood was collected 45 days later and the cells were stained for B220, CD19, and AA4.1. Of note, in the poly(I)-poly(C) treated BAFF-R^{FL/FL}/MX-cre mice, splenic B cell numbers were reduced by about 50% (15×10⁶±3×10⁶ relative to 28×10⁶±5×10⁶ in the control mice, data not shown). FIGS. 4E-G show estimation of depletion efficiency. To determine depletion efficiency, inventors generated MX-Cre BAFF-R floxed mice that were transgenic for the EYFP-Cre reporter system that was targeted into the ROSA26 locus. Splenic cells from poly(I)-poly(C)-treated (FIGS. 4F-G), or untreated (FIG. 4E) mice were analyzed for YFP and B220. Expression of the YFP was an indication for Cre expression and activation and served as an estimate for the deletion efficiency of the BAFF-R floxed allele.

FIGS. 5A-M are graphs depicting a maintained young-like B lineage in old mice following ablation of BAFF-R. Old C57B16 BAFF-R^{FL/FL}/MX-cre and young and old control C57B16 BAFF-R^{FL/+}/MX-cre mice were treated with poly(I)-poly(C) to ablate the floxed-BAFF-R allele. After 90 days, BM was analyzed for B lymphopoiesis. FIGS. 5A-I show BM cells which were stained for B220, IgM, CD43, and AA4.1 and analyzed by FACS. The IgM vs. AA4.1 plots (FIGS. 5B, 5E and 5H) were gated for B220+ cells, and the B220 vs. CD43 plots (FIGS. 5C, 5F and 5I) were gated on IgM^{neg} cells. FIGS. 5J-M show quantification of B-cell subsets in the BM. The number of total B cells was calculated from the % B cells (measured by FACS) multiplied by the number of viable cells collected from one femur and one tibia. The relative proportion of each B-cell subset was determined in the B220-gated population. Shown are representative results of young (n=3) and old (n=3) control BAFF-R^{FL/+}/MX-cre mice and two different Old BAFF-R^{FL/FL}/MX-cre mice (from n=3).

FIGS. 6A-N depict renewed B lymphopoiesis and rejuvenated peripheral repertoire in aged mice following B-cell depletion. FIGS. 6A-C show kinetics of B-cell return after depletion. Old B10D2 wt mice (n=5) were subjected to three rounds of

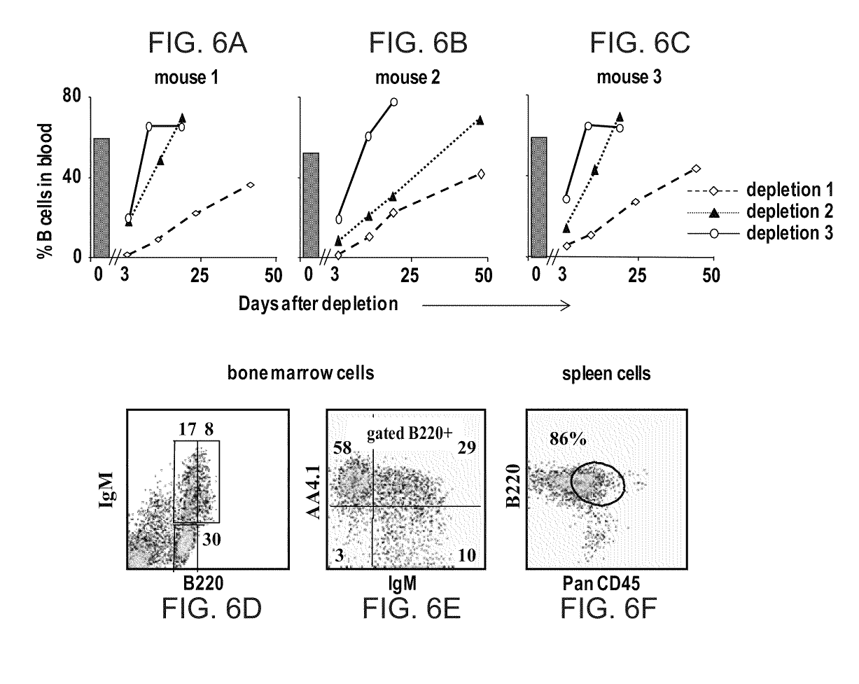

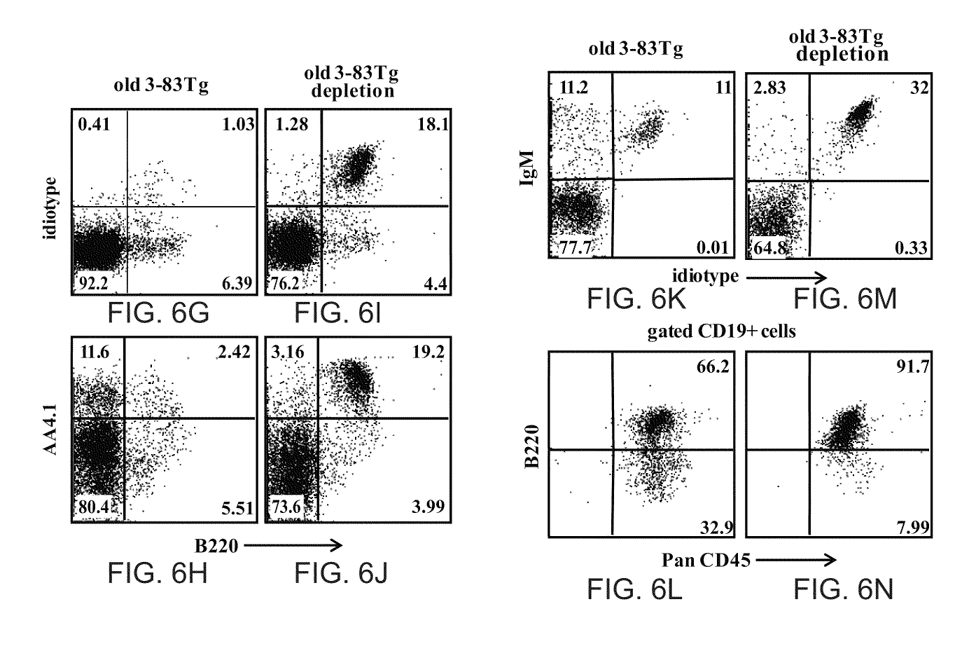

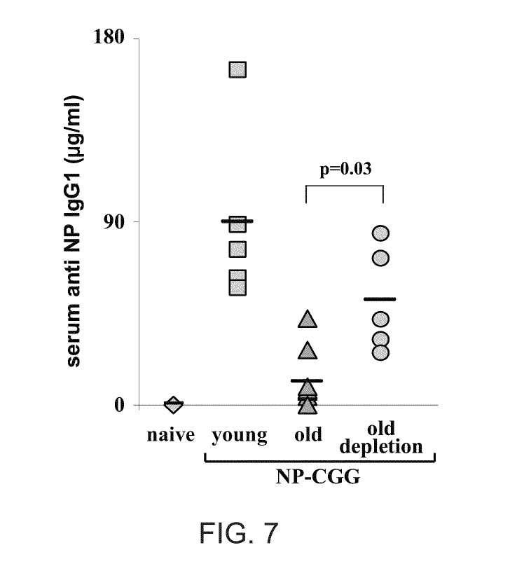

B-cell depletion by specific antibodies. The second and the third rounds were initiated only after more than 80% of the initial B-cell numbers had been restored. Blood samples were collected at various times after depletion and analyzed for B-cell frequency. The time-course of B-cell return after each depletion cycle is shown for three individual mice. The histogram in each plot corresponds to the B-cell frequency in peripheral blood before the first depletion. FIGS. 6D-F show the B lineage cells from the BM (FIGS. 6D-E) and spleen (FIG. 6F) of mice subjected to three rounds of depletion as was analyzed by FACS. BM cells (FIGS. 6D-E) were stained for B220, IgM, and AA4.1. The analysis of IgM and AA4.1 (FIG. 6E) was conducted on gated B220+ cells. Spleen cells (FIG. 6F) were stained for IgM, B220, and PanCD45. The analysis for B220 and PanCD45 was conducted on gated IgM+ cells. Representative plots of five mice are shown. FIGS. 6G-N show that B-cell depletion in old 3-83 Tg mice revives B lymphopoiesis in the BM and rejuvenates the peripheral repertoire. Old B10D2 3-83 Tg mice (confirmed to have an old-like B-cell phenotype by blood-sample staining) were treated for B-cell depletion using specific antibodies. After 65 days, the BM and spleen were analyzed for the B lineage by flow cytometry. FIGS. 6G-J, BM cells were stained and analyzed for the expression of IgM, B220, AA4.1, and idiotype. FIGS. 6K-N, spleen cells were stained and analyzed for IgM, B220, PanCD45, and idiotype. The analysis for B220 and PanCD45 was conducted on gated CD19+ cells. The plots shown are representative of 4 mice in each group.

FIG. 7 depicts an increased anti-NP IgG1 response in old mice with a rejuvenated peripheral repertoire. Old C57B16 wt mice that were untreated or subjected to one round of B-cell depletion and young untreated mice were immunized i.p. with NP-CGG. The amount of anti-NP-specific IgG1 antibodies in the serum was determined by ELISA 7 days later, using an IgG1 standard curve for reference. The results for individual mice and the mean for each group are shown. Student's t-test was conducted to examine statistical significance between the means (n=5) of the antibody titers of different groups.

### DESCRIPTION OF SPECIFIC EMBODIMENTS OF THE INVENTION

The present invention, in some embodiments thereof, relates to methods of altering peripheral B cell populations and, more particularly, but not exclusively, to the use of same for improving immune competence.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have surprisingly uncovered that senescence in the B lineage can be reversed by altering B cell homeostasis. Thus, in vivo depletion of B cells in aged mice revives B lymphopoiesis in the bone marrow, rejuvenates the peripheral repertoire and restores B cell competence to mount an antibody response to new antigenic challenges.

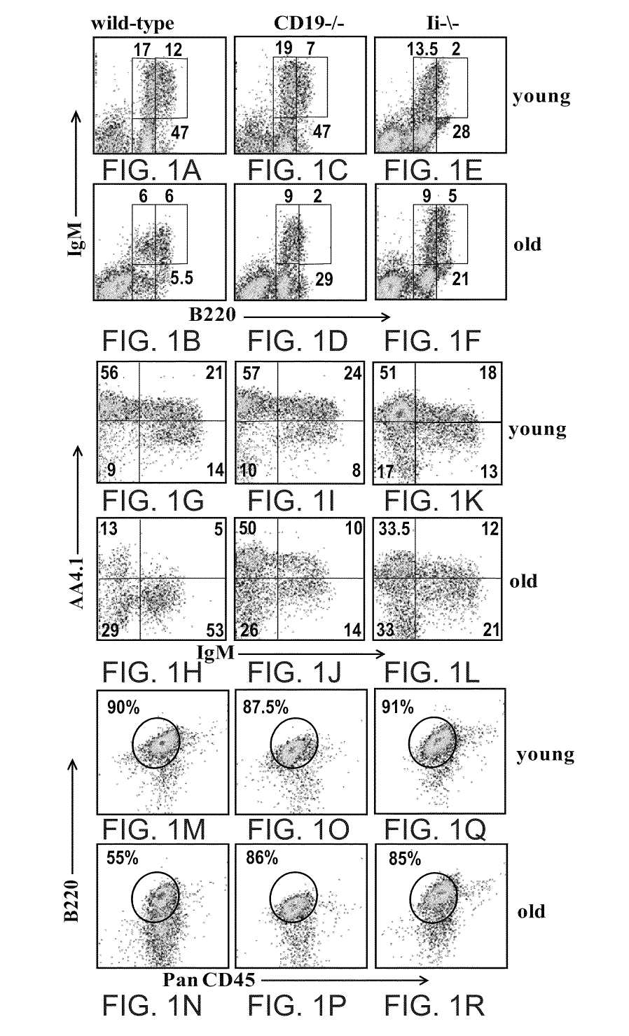

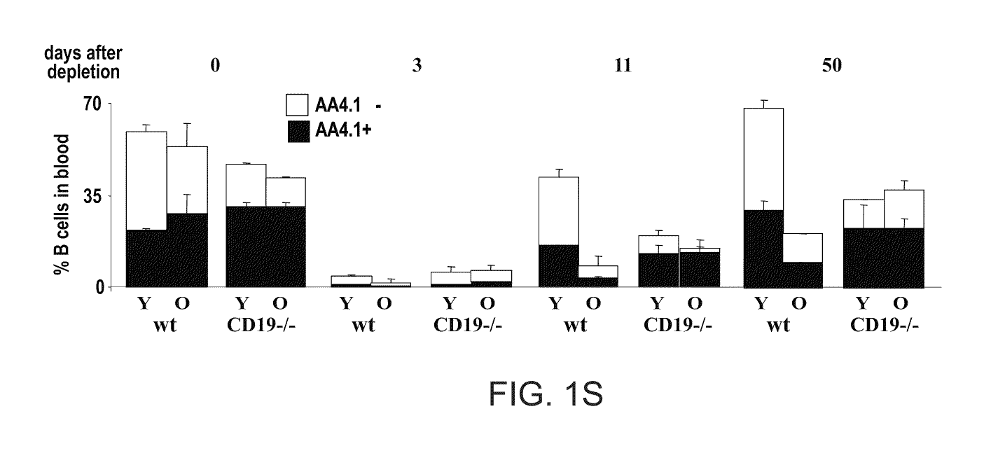

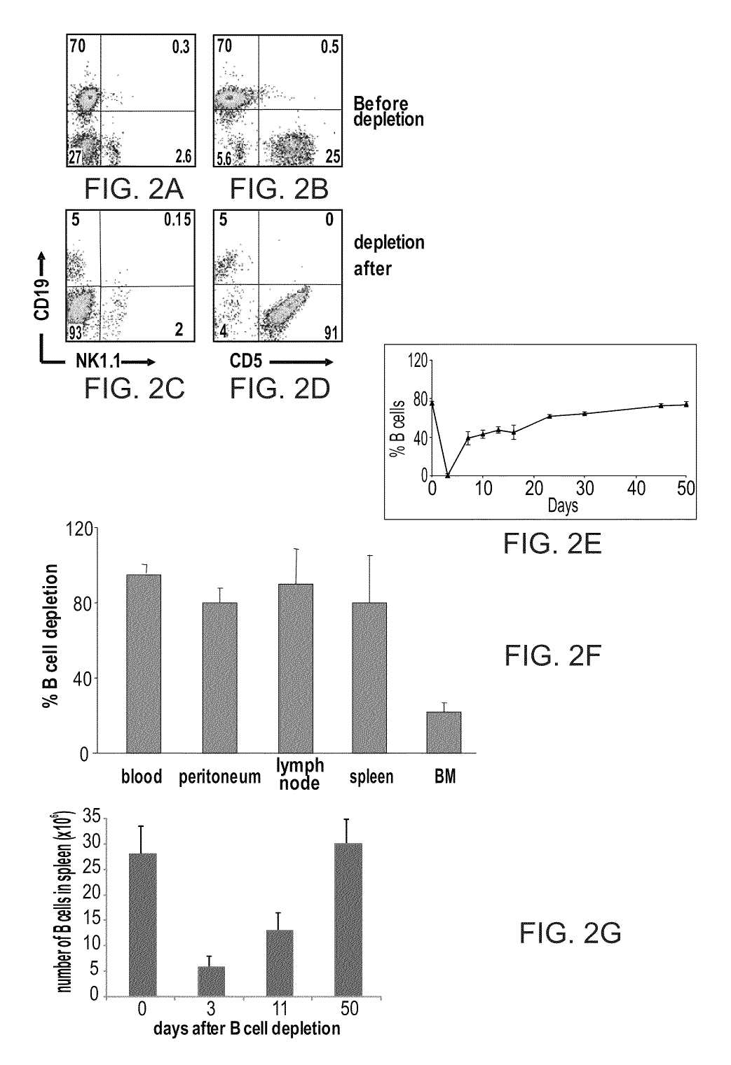

As is shown hereinbelow and in the Examples section which follows, the present inventors have uncovered through laborious experimentation that depletion of peripheral B cells such as by specific antibodies (FIGS. 1S and 2A-G) leads to nearly complete reconstitution of B cells in young mice within two weeks (FIGS. 1S and 2E) whereas in aged mice only partial reconstitution was observed after 50 days. However, repeated depletions in aged mice lead to an effectively revitalization of B lymphopoiesis (in the BM of aged mice) and shorten the reconstitution rate to one comparable to that of young mice (FIGS. 1S and 6A-F). Thus, altering peripheral B cell populations (i.e. by depleting peripheral B cells) induces generation of B cells in the bone marrow (i.e. B lymphopoiesis). These newly generated B cells migrate to the peripheral organs and lead to a rejuvenated B cell response and antibody production (FIG. 7). Taken together, all these findings substantiate altering B cell homeostasis, by depletion of peripheral B cells, for improving immune competence.

Thus, according to one aspect of the present invention there is provided a method of altering peripheral B cell populations in a subject in need thereof, the method comprising administering to the subject a therapeutically effective amount of an agent capable of depleting peripheral B cells in the subject, and wherein the subject does not have a hematologic cancer or an autoimmune disease, thereby altering the peripheral B cell populations in the subject.

The term “peripheral B cell” as used herein refers to circulating B lymphocytes which are present in the peripheral organs including e.g. the blood and lymphatic systems. According to a specific embodiment, the peripheral B cells are not in the bone marrow. Peripheral B cells, as used herein, refers to any subset of B cells including immature B cells, transitional B, mature B cells, plasma B cells and memory B cells.

The phrase “peripheral B cell populations” as used herein refers to the different subsets of B cells in the peripheral organs e.g. mature B cells, immature B cells, transitional B cells, naïve B cells, memory B cells, plasma B cells, marginal zone B cells, and B-1 B cells or any subsets therefrom.

The phrase “altering peripheral B cell populations” as used herein refers to a change in number in at least one subset of B cell populations in the host peripheral organs, either an increase (e.g., at least 5%, 10%, 15%, 20%, 30%, 50%, 100%, 200%, 250%, 400% or more) or a decrease (e.g., at least 5%, 10%, 15%, 20%, 30%, 50%, 100%, 200%, 250%, 400% or more) in the B cell subset levels. Altering is typically determined with respect to an untreated subject (i.e., who was not subject to altering peripheral B cell populations) or with respect to the subject prior to treatment, and may be determined by any method known to one of ordinary skill in the art, as for example, by FACS analysis of cellular markers (e.g. CD20, CD19, IL-7 receptor) expressed by B cell populations, by serum protein electrophoresis measuring production of antibodies or by fluorescent microscopy or scanning electron microscopy (SEM) measuring morphological features of the different B cell populations.

As used herein, the phrase “subject in need thereof” refers to a mammal, preferably a human being, male or female that is in need immune reconstitution. Typically the subject is an adult subject (e.g. over 40 years old), an immune compromised subject, or a combination of both. The subject may be immune compromised due to a disorder or a pathological or undesired condition, state, or syndrome, or a physical, morphological or physiological abnormality which is amenable to treatment via altering peripheral B cell populations. Examples of such disorders are provided further below.

As mentioned, according to a specific embodiment, the subject is at least 40 years old. According to other embodiments of the present invention, the subject is at least 50, 60, 70, 80, 90 or more years old. The subject may be generally healthy (whereby treatment is effected for prophylactic purposes) or immune compromised such as detailed below.

Thus, a number of diseases and conditions, which involve B cells, can be treated using the present teachings. Examples of such diseases and conditions are summarized infra.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998;7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyper-acute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include, but are not limited to, carcinoma, blastoma and sarcoma. Particular examples of cancerous diseases but are not limited to: Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

Immune Deficiency Diseases

Examples of immune deficiency diseases include, but are not limited to, acquired immunodeficiency syndrome (AIDS), X-Linked Agammaglobulinemia (XLA), Common Variable Immunodeficiency (CVID)/Hypogammaglobulinemia, Hyper-IgM Syndrome, Selective IgA Deficiency, IgG Subclass Deficiency, Severe Combined Immunodeficiency (SCID), Wiskott-Aldrich Syndrome, DiGeorge Syndrome and Ataxia-telangiectasia.

As mentioned, the method of the present invention is effected by administering to the subject a therapeutically effective amount of an agent capable of depleting peripheral B cells in the subject.

As used herein a “therapeutically effective amount” refers to an amount sufficient for depleting peripheral B cells and inducing generation of new B cells in the bone marrow.

As used herein, the term “depleting peripheral B cells” refers to reducing the levels of peripheral B cells by at least 10%, at least 20%, at least 30%, at least 40%, at least 50%, at least 60%, at least 70%, at least 80%, at least 90%, at least 95%, or at least 100%. Typically, depleting peripheral B cells refers to killing thereof, phagocytosis thereof, inducing apoptosis thereof or inducing growth arrest thereof. Measuring depletion of peripheral B cells may be carried out by any method known to one of ordinary skill in the art, as for example, by apoptosis assays [e.g. caspase assays, TUNEL and DNA fragmentation assays, cell permeability assays, Annexin V assays, protein cleavage assays, mitochondrial assays and ATP/ADP assays, as described in detail in worldwideweb(dot)apoptosisworld(dot)com/ApoptosisAssays(dot)html] and growth arrest assays [e.g. ELISA, Immunoflourescence or Western blot, using for example, Growth arrest-specific (GAS2) Monoclonal Antibody (4E11) available from Assay Designs, Mich., USA].

It will be appreciated that B cell depletion may be effected by any method known to one of ordinary skill in the art. For example, B cell depletion can be effected directly by causing B-cell cell death (e.g. via antibodies or cytotoxins) or alternatively by depleting a factor essential for B cell survival. The factor can be endogenous to the B cells or exogenous to the B cell (i.e., a B cell trophic factor) secreted from, for example, other immune cells e.g. macrophages.

For example, any agent which may deplete peripheral B cells may be used in accordance with the present teachings. The agent may be a nucleic acid agent, which interferes with transcription and/or translation of a B cell factor (e.g., RNA silencing agents, Ribozyme, DNAzyme and antisense) either directly (i.e. within the B cell itself) or indirectly (i.e. by affecting other cells). Alternatively, the agent may be a polypeptide agent (e.g., an antibody, an antagonist, a fusion protein or an enzyme) which leads directly or indirectly to depletion of peripheral B cells. Yet, alternatively, the agents can be a small molecule which is targeted specifically to the B-cell and induces killing thereof. Still alternatively, any combination of the aforementioned agents can be used.

Following is a summary of agents which can be used in accordance with the present teachings. The description is not intended to be limiting.

An example of an agent capable of depleting peripheral B cells is an antibody capable of specifically binding B cells and killing same or an antibody capable of neutralizing a B cell trophic factor by binding to same or to an effector thereof, as is described below. Preferably, the antibody specifically binds at least one epitope of a B cell cellular protein. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

It will be appreciated that the present invention contemplates the use of any antibody that induces B cell depletion directly (e.g. by antibody-dependent cellular cytotoxicity (ADCC), by complement-dependent cytotoxicity (CDC), by phagocytosis, and by apoptosis) or indirectly (e.g. via factors which are vital for B cell survival). Furthermore, the antibody may be conjugated to other active motifs, as for example, to a radioactive isotope (i.e. monoclonal antibody radioimmunotherapy e.g. Ibritumomab tiuxetan).

Specifically, the present invention contemplates the use of an anti-B cell antibody to deplete peripheral B cells. For example, a suitable anti-B cell antibody can be an antibody targeting any B cell membrane receptor e.g. an anti-CD20 monoclonal antibody [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Genmab), TRU-015 (Trubion) and IMMU-106 (Immunomedics)], an anti-CD22 antibody [e.g. Epratuzumab, Leonard et al., Clinical Cancer Research (2004) 10: 5327-5334], an anti-CD79a antibody, an anti-CD27 antibody, or an anti-CD19 antibody (e.g. U.S. Pat. No. 7,109,304).

It will be appreciated, that an anti-B cell antibody may be an antibody which inhibits binding and activation of a B cell receptor. Such antibodies include the anti-BAFF-R antibody (e.g. Belimumab, GlaxoSmithKline), the anti-TACI antibody (e.g. Belimumab, GlaxoSmithKline) and the anti-BCMA antibody (e.g. Belimumab, GlaxoSmithKline).

As mentioned, the antibody used to deplete peripheral B cells may be an antibody targeting a B cell survival factor or a cytokine imperative for B cell function or an effector thereof (e.g., a receptor which binds the aforementioned factor). For example, the anti-Blys (BAFF) monoclonal antibody (e.g. Belimumab,

GlaxoSmithKline), the anti-APRIL antibody (e.g. anti-human APRIL antibody, ProSci inc.), the anti-IL-6 antibody [previously described by De Benedetti et al., J Immunol (2001) 166: 4334-4340 and by Suzuki et al., Europ J of Immunol (1992) 22 (8) 1989-1993, fully incorporated herein by reference], the anti-IL-7 antibody (R&D Systems, Minneapolis, Minn.) or the SDF-1 antibody (R&D Systems, Minneapolis, Minn.).

As mentioned above, depletion of peripheral B cells may also be achieved by the use of fusion proteins which block activation of B cell receptors. For example, a fusion protein composed of the extracellular ligand binding portion of TACI which blocks activation of TACI by April and BLyS (e.g. Atacicept, Merck) or a fusion protein composed of the extracellular ligand-binding portion of BAFF-R which blocks activation of BAFF-R by BLys (e.g. BR3-Fc, Biogen and Genentech). Such fusion proteins can be generated using methods known in the art, such as recombinant DNA technology as is described in details herein below.

Alternatively, fusion proteins can be generated using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like.

Depleting peripheral B cells can be also achieved using an agent for RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 by duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the target mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable siRNA which may be used in accordance with the present teachings may be one which targets CD20 mRNA (which is coding for the CD20 protein). An example of such a siRNA is CCACTCTTCAGGAGGATGT (SEQ ID NO: 1) as was previously described by Li et al., J Biol Chem. (2003) 278:42427-34. Other suitable siRNAs can be e.g. CD20 siRNA (Santa Cruz, sc-29972), CD22 siRNA (Santa Cruz, sc-29807), BAFF siRNA [Haiat et al., Immunology (2006) 118(3): 281-292] or kappa light chain siRNA.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIs1, TAT(48-60), pVEC, MTS, and MAP.

Another agent capable of depleting peripheral B cells is a DNAzyme molecule capable of specifically cleaving an mRNA transcript or DNA sequence of the target sequence. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Depleting peripheral B cells can also be effected using an antisense polynucleotide capable of specifically hybridizing with an mRNA transcript encoding the target sequence.

Design of antisense molecules which can be used to efficiently downregulate a target sequence, and thus deplete peripheral B cells, must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

For example, a suitable antisense oligonucleotides may be one which targets kappa light chain mRNA (which is coding for the kappa light chain protein).

Another agent capable of depleting peripheral B cells is a ribozyme molecule capable of specifically cleaving an mRNA transcript encoding a B cell specific protein (e.g. CD20). Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

B cell depletion can also be effected by specifically expressing in the B cell a nucleic acid agent encoding a cytotoxin or alternatively contacting the B cell with a protein encoding a toxin fused to B cell targeting agent (e.g., anti-CD20 antibody). Thus, upregulating within a B cell expression of a cytotoxic polypeptide (e.g. Pseudomonas exotoxin, Diphtheria toxin or Ricin toxin), expressed under regulation of a B cell specific promoter, can effectively lead to B cell depletion (e.g. via apoptosis or growth arrest).

Examples of B cell specific promoters include, but are not limited to, the CD20/B1 antigen promoter [Rieckmann P. et al., J Immunol. (1991) 147(11):3994-3999], the pre-B- and B-cell-specific mb-1 promoter [Travis A. et al., Mol Cell Biol. (1991) 11(10:5756-5766], the CD19 promoter [Kozmik et al., Mol Cell Biol. (1992) 12(6): 2662-2672 1, the CD22 promoter [Wilson et al., J Immunology, (1993) 150(11) 5013-5024], the Kappa promoter [Kossakowska and Urbanski, Immunology (1989) 66(3): 328-334], the lambda promoter [Martensson and Melchers, International Immunology (1994) 6(6) 863-872] and the immunoglobulin heavy-chain promoter [LeBowitz et al., Genes Dev. (1988) 2(10):1227-1237].

Alternatively, B cell depletion can be effected by reducing the survival or activity of a B cell trophic cell (e.g., monocyte, macrophage, stromal cell, astrocyte or synoviocyte) by, for example, contacting the B cell trophic cell with a nucleic acid agent encoding a cytotoxin (e.g. Pseudomonas exotoxin, Diphtheria toxin or Ricin toxin) under regulation of a specific promoter. It will be appreciated that one of ordinary skill in the art would know how to choose such a promoter. Alternatively, the B cell trophic cell may be contacted with a protein encoding a toxin fused to a targeting agent (e.g., specific target cell antibody such as anti-CD14 antibody for macrophages). Reducing the survival or activity of the B cell trophic cell as mentioned can effectively lead to B cell depletion (e.g. via lack of essential B cell survival factors).

Thus, the nucleic acid agents of the present invention can be contacted with B cells or B cell trophic cells (e.g., monocytes, macrophages, stromal cells, astrocytes or synoviocytes). This can be effected by in vivo gene therapy or ex-vivo gene therapy.

Typically, the present invention envisages in vivo treatments with the aforementioned agents or ex vivo treatments, whereby B cells are retrieved (e.g., by leukophoresis) from the subject treated with the aforementioned agents and returned to the subject.

Any of the above mentioned nucleic acid agents can be ligated into a nucleic acid expression construct and placed under the regulation of a cis-regulaotry element such as a promoter. B cell specific promoters were described above. Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M, et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of toxins since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Each of the agents described hereinabove for depleting peripheral B cells can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agents described hereinabove for depleting peripheral B cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (agents for depleting peripheral B cells) effective to deplete peripheral B cells of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide adequate levels of the active ingredient as to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that the agents of the present invention may be administered for a chronic treatment (i.e. a disease or condition that is long-lasting or recurrent) or for an acute treatment (i.e. a disease or condition which has a rapid onset or a short course).

The agents of the present invention will be given for a sufficient amount of time to enable depletion of peripheral B cells without causing a complete immune deficiency in the subject (i.e. in which the immune system does not function). Thus, it is advisable to draw a base-line blood sample from each subject prior to administration of the agents of the present invention. Furthermore, once a subject received the agents of the present invention, it is advisable that they return for follow-up evaluation, which include, for example, hematologic and chemical tests for safety.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that in order to deplete peripheral B cells and improve immune competence, additional factors may be administered to the subject. For example, G-CSF may be administered prior to, concomitantly with, or following administration of the above described agents (e.g. anti-CD20 antibody) in order to stimulate the bone marrow to produce stem cells. Likewise, other growth factor and/or cytokines may be administered to the subject including, but not limited to, IL-6, IL-7 and SDF-1.

In addition, vitamin and mineral additives may be administered to the subjects, especially elderly subjects, in order to improve immune competence and health. Such additives include, but are not limited to, Vitamin A, Vitamin B, Vitamin D, Vitamin E, Vitamin K, Riboflavin, iron, folate, niacin and calcium.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Experimental Mice

B10D2 mice were used which were normal or deficient in CD19 (CD19−/−) [as was previously described in Rickert et al., Nature (1995) 376, 352-355] or were 3-83 Tg [as was previously described in Russell et al., Nature (1991) 354, 308-311].

C57B16 mice were used which were normal or deficient of invariant chain (Ii−/−) [as was previously described in Shachar and Flavell, Science (1996) 274, 106-108], or carrying a targeted BAFF-R floxed locus (BAFF-R^{f}) [as was previously described in Sasaki et al., J. Immunol. (2004) 173, 2245-2252], or transgenic for MX-Cre [as was previously described in Kuhn et al., Science (1995) 269, 1427-1429], or transgenic for the EYFP-Cre reporter system [as was previously described in Srinivas et al., BMC Dev Biol (2001) 1, 4].

All mice were housed and bred at the animal facility of the Faculty of Medicine, Technion, and all studies were approved by the committee for the supervision of animal experiments.

For all experiments, old mice were considered as more than 20 months old and young mice were considered as 3-4 month old.

Depletion of B Cells

In order to deplete B cells in vivo, mice were injected intraperitoneal (i.p.) with the following mixture of monoclonal antibodies at 150 μg/mouse each: rat anti-mouse CD19 (clone 1D3), rat anti-mouse B220 (clone RA36B2), and mouse anti-mouse CD22 (clone CY34). After 48 hours, the mice were injected with 150 μg/mouse secondary antibody mouse anti-rat kappa (clone TIB216).

Injected mice were bled from tail vain at different time intervals to determine depletion efficiency and the kinetics of B cell return by flow cytometry. In some experiments, mice were sacrificed to determine depletion efficiency by flow cytometry and by absolute numbers in different lymphoid organs.

Flow Cytometry

Single cell suspensions of from BM, spleen, lymph node, peritoneal cavity and peripheral blood were stained for surface markers expression using FITC-, PE-, APC and biotin-conjugated antibodies, followed with streptavidin PerCP. The following antibodies were used: anti-IgM (Caltag), anti-B220 (Southern Biotechnology Associates, SBA), anti-PanCD45 (SBA), anti-AA4.1 (eBioscience), anti-CD19 (eBioscience), anti-NK1.1 (Biolegend), anti-CD5 (Pharmingen), anti-3-83 idiotypic Ab (54.1 the inventor's hybridoma), anti- CD43 (Pharmingen) and anti-YFP.

Data for three and four-color analysis were collected on a FACSCalibur™ (BD Biosciences, Immunocytometry Systems, Mountain View, Calif., USA) and analyzed using CELLQuest™ or FlowJo software.

Poly(I)poly(C) Administration

To activate the MX-Cre in vivo for ablation of lox-p floxed loci, mice received i.p. injections of three doses of poly(I)-poly(C) (400 μg each) (InvivoGene, San Diego, Calif.) on days 0, 3, and 6. Depletion efficiency was determined by flow cytometry using the EYFP-Cre reporter system as previously described [Srinivas et al., supra].

Immunization

Old C57B16 wt mice that were untreated or subjected to one round of B-cell depletion (with antibodies) and young untreated mice were immunized i.p. with NP-CGG (50 μg/mouse) in alumn adjuvant. 7 days later, mice were bled from tail vain and anti NP IgG1 antibodies in serum were determined by standard ELISA using NP-BSA coated plates and using an IgG1 standard curve for reference (see below).

ELISA (Enzyme-Linked Immunosorbent Assay)

96-well flat bottom plates were coated with 50 μl NP-BSA overnight at 4° C. After removing the unbound antigen, the wells were blocked with blocking buffer for 1-3 hours at room temperature. Serums obtained from the different treated mice (in serial two-fold dilutions) were applied to the plates and incubated for 1-3 hours at room temperature. The wells were then washed 3 times with washing buffer (PBS with 0.05% Tween) and were incubated for 1 hour with 50 μl goat anti mouse IgG1 detecting antibody conjugated to biotin (diluted 1:2500 in blocking buffer) at room temperature. After washing, 50 μl sterptavidine-HRP (diluted 1:2500 in blocking buffer) was added for 1 hour. Plates were then washed and 50 μl TMB solution was added to each well and the reaction was stopped by stop solution (2M H₂SO₄). Plates were read using an ELISA Reader at 450 nm with 630 nm as the reference. Amounts of IgG1 anti NP were calculated using an IgG1 standard curve as a reference.

### Example 1

### Chronic Homeostatic Demands Block Senescence in B Lymphopoiesis

To examine the mechanism by which the B lineage enters senescence, inventors used mice deficient in CD19 (CD19−/−) or invariant chain (Ii−/−). In these mouse models, B-cell development in the BM was almost unperturbed, but B-cell maturation and survival in the periphery was impaired owing in part to a defective T-cell response. Thus, these mouse lines had a chronic B-cell deficiency (reduced by 30-40%) in the periphery. Inventors hypothesized that if senescence of the B lineage was a progressive and not reversible process, then the CD19−/− and Ii−/− mice should gradually lose their peripheral B cells and become B-cell-less mice as they aged. If, however, senescence of the B lineage was due to homeostatic pressures from the long-lived peripheral B cells, then B lymphopoiesis in the CD19- or Ii-deficient mice, which lacked long-lived B cells, should not enter senescence.

As shown in FIGS. 1A-S, B lymphopoiesis in the mutant mice with chronic B-cell deficiency did not become senescent, supporting the second hypothesis. First (data not shown), inventors found that the total number of B cells in the BM of old CD19−/− and Ii−/− mice was 7-8 fold higher than that in the BM of old wild type (wt) mice (17×10⁶±4×10⁶ for CD19−/− mice and 14×10⁶±2.8×10⁶ for Ii−/− mice, relative to 2.1×10⁶±1×10⁶ for wt mice), and similar to their numbers in the BM of young mice (22×10⁶±4.8×10⁶). Second, while the frequencies of pro/pre (B220^{lo}/IgM−) and immature (B220^{lo}/IgM+) B cells dropped dramatically in old wild-type (wt) mice, old CD19−/− and Ii−/− mice retained high frequencies of these cell populations in their BM (FIGS. 1A-F). This finding was further supported by an analysis of the BM B cells for the early developmental marker AA4.1 (FIGS. 1G-L). Third, old mice deficient in CD19 or Ii were still deficient in B cells in the periphery as revealed by 30-40% reduction in the number of splenic B cells (14×10⁶±5 and 16×10⁶±6, respectively, relative to 26×10⁶±4 in old wt mice, and FIGS. 1M-R). However, the peripheral B cells of these old B-cell deficient mice retained a young-like phenotype. In the old wt mice, about 50% of the splenic B cells acquired the antigen-experienced phenotype PanCD45+/B220^{lo} whereas only about 15% of such cells were found in the spleens of old CD19−/− and Ii−/− mice (FIGS. 1N, 1P, 1R).

To determine the B cell production capacity of the BM, inventors analyzed the kinetics of B-cell output. To examine this, inventors used a mixture of antibodies (see general materials and experimental procedures section above) to deplete the B cells in vivo and followed the auto-reconstitution of the B cells by analyzing peripheral blood samples over time. Using this treatment, inventors established depletion of 80-90% of the B cells as analyzed in peripheral blood, spleen, peritoneal cavity and lymph node (FIGS. 2A-D and 2F). The results in FIGS. 2F-G and 1S illustrated that the B-cell depletion (on day 3) reached more than 80% in all mice. As expected, inventors found a profound difference in the kinetics of B-cell return between the young and old wt mice. While young wt mice rapidly reconstituted the peripheral B cells, almost reaching their original level by 11 days (FIG. 1S), old wt mice reconstituted only 50-60% of their peripheral blood B cells 50 days after depletion. A high frequency of AA4.1+B cells indicated that most of the reconstituted cells were newly generated (FIG. 1S).

The difference in the reconstitution rate between young and old wt mice has been attributed to the decreased B lymphopoiesis in the BM of old mice. However, as depicted herein, young and old CD19−/− mice showed a rapid reconstitution of their peripheral B cells, at an indistinguishable rate (FIG. 1S), indicating that their rate of B lymphopoiesis in the BM did not drop with age. This finding was further supported by the observation that many of the cells in the young and old CD19−/− mice were newly generated AA4.1+ cells (FIG. 1S). It is important to note that since CD19−/− B cells have a shortened half-life due to impaired maturation, the rate of peripheral B-cell auto-reconstitution in CD19−/− mice was expected to be lower than in young wt mice. Hence, inventors concluded that a chronic B-cell deficiency prevented B lymphopoiesis from entering senescence and maintained a young-like peripheral B-cell phenotype.

### Example 2

### Peripheral Repertoire Does Not Age Upon Chronic Homeostatic Demands

Since the diversity of the peripheral B-cell repertoire is profoundly reduced with aging, inventors next tested whether the development of an age-dependent limited repertoire was prevented in mice deficient in CD19. Cambier and colleagues [Johnson, Rozzo and Cambier, J. Immunol. (2002) 168, 5014-5023] used an immunoglobulin transgenic (Ig-Tg) mouse model (3-83 Tg mice) to report age-associated changes in the B-cell repertoire. This peripheral repertoire changed with aging, and in old 3-83 Tg mice, it was dominated by B cells that expressed endogenous receptors. According to Russell et al. [Russell et al. Nature (1991) 354, 308-311], in young 3-83 Tg mice, about 90% of the splenic B cells expressed the transgenic receptor.

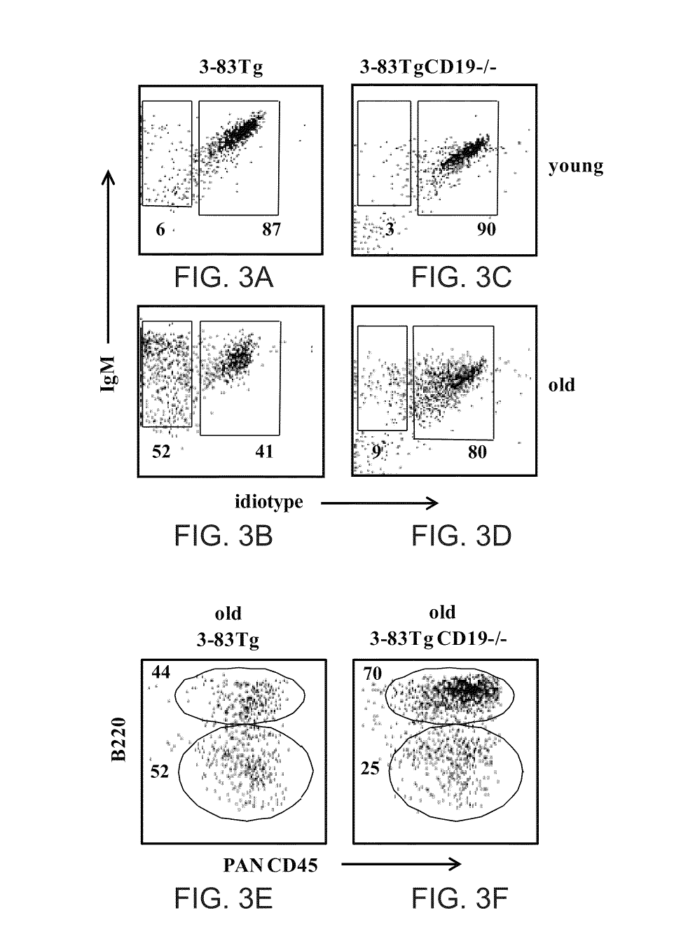

In sharp contrast, the inventors of the present invention found that the development of this old-like repertoire was prevented in 3-83 Tg mice that were deficient in CD19 (FIGS. 3A-F), in which the peripheral B cells were chronically reduced by 30-40%. Thus, in the old 3-83 Tg CD19−/− mice, most of the B cells in the spleen (about 80%) retained expression of the transgenic receptor, compared to only about 40% in the old 3-83 Tg mice (FIGS. 3B and 3D). Also, only 20-25% of the splenic B cells in the old 3-83 Tg CD19−/− mice expressed the old-like phenotype PanCD45+/B220^{lo}, compared to more than 50% in the old 3-83 Tg mice (FIGS. 3E-F). Hence, inventors concluded that a chronic B-cell deficiency prevented the senescence of B lymphopoiesis and the B-cell repertoire.

### Example 3

### Limiting BAFF Signaling Stimulates Homeostatic Demands for B Cells and Prevents Senescence in the B Lineage

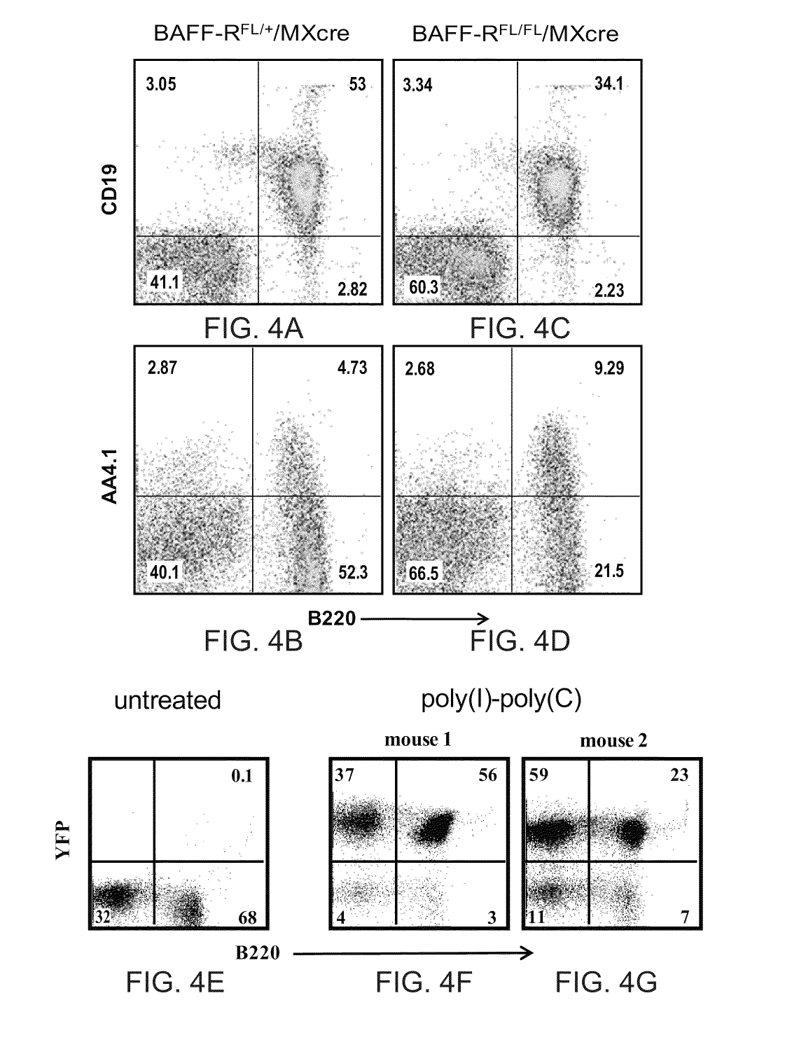

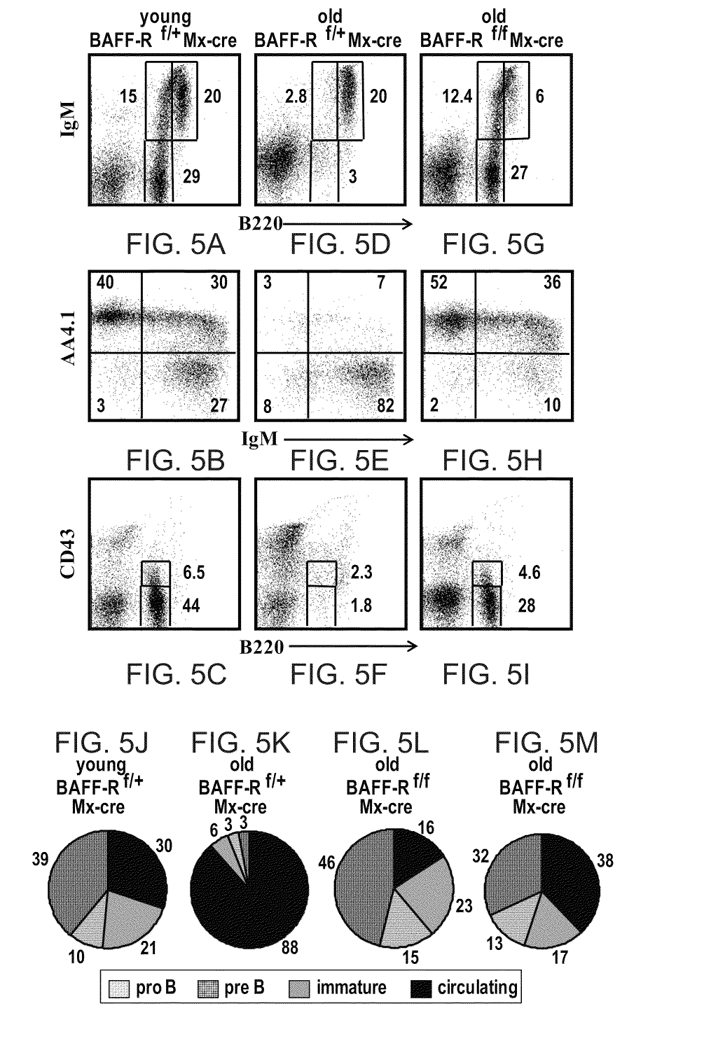

Since lack CD19 or Ii perturbs the normal function of the B cells, inventors decided to verify these observations under physiological conditions. To do so, inventors used mice homozygous for a targeted loxP-flanked BAFF-R allele (BAFF-R^{f/f}) and transgenic for cre-recombinase driven by the interferon promoter (Mx-cre). BAFF-BAFF-R signaling is an essential survival factor for mature B cells, but is dispensable for B lymphopoiesis in the BM. In these mice, administration of the interferon inducer poly(I)-poly(C) resulted in the ablation of BAFF-R (depletion efficiency in the spleen 80-90% as determined by EYFP-Cre-reporter system, FIGS. 4E-G), the depletion of about 50% of the B cells as measured in the peripheral blood and spleen, and the appearance of many newly generated B220+/AA4.1+ B cells (FIGS. 4A-D). In old BAFF-R^{f/f} Mx-cre mice, the peripheral B-cell deficiency that was induced upon ablation of BAFF-R stimulated significant B lymphopoiesis in the BM that was similar to the B lymphopoiesis of young mice (FIGS. 5A-I). Inventors found a high frequency of proB (B220+/CD43+/IgM−), preB (B220+/CD43−/IgM−), and immature B (B220^{lo}/IgM+) cells in the BM of old BAFF-R^{f/f} Mx-cre mice, but not of old control BAFF-R^{f/+} Mx-cre mice (FIGS. 5A-I). Quantification revealed a similar number of B cells in the BM of young mice and of old BAFF-R^{f/f} Mx-cre mice treated with poly(I)poly(C) (20×10⁶±2.4×10⁶ and 15×10⁶±3.6×10⁶, respectively), with about 75% of the cells being newly generated and residing in the BM (proB, preB, immature) with only 25% circulating (B220^{hi}/IgM+/AA4.1−) (FIGS. 5J-M). In contrast, about 10 fold less B cells were found in the BM of the old control BAFF-R^{f/+} Mx-cre mice (2×10⁶±5×10⁵ cells), and 88% of them were circulating (FIGS. 5J-M).

Collectively, these results indicated that the immunosenescence of B lymphopoiesis in the BM reflected homeostatic pressures that are set by the demand for B cells in the periphery. In cases of continuous demands, such as in CD 19−/− or Ii−/− mice, or an acutely induced demand, such as following the ablation of BAFF-R, B lymphopoiesis does not enter senescence, thereby to ensure a continuous flow of B cells to the periphery.

### Example 4

### Induction of Homeostatic Demands Upon Active B Cell Depletion Rejuvenates the B Lineage in Old Wild-Type Mice

As the results above indicated that the senescence of B lymphopoiesis reflects homeostatic demands, inventors next tested whether altering the B-cell homeostasis could revive B lymphopoiesis in aged mice. To test this, old wt mice were subjected to multiple rounds of antibody-mediated B-cell depletion and were assessed for the auto-reconstitution rate of B cells as measured in the peripheral blood.

As shown in FIGS. 6A-C, peripheral reconstitution after the first B-cell depletion took more than 50 days. This slow reconstitution rate reflected the poor B lymphopoiesis of the aged BM. However, the reconstitution rate of the B cells after the second round of depletion increased by more than 70%, and full reconstitution was established within 18-30 days. This reconstitution rate improved to be faster by more than 85% after the third depletion, and complete B-cell reconstitution was observed within 8 days (FIGS. 6A-C), which is similar to the rate of B-cell return in young wt mice (FIG. 1S).

The profound increase in the rate of B-cell return after multiple depletions suggested that the alteration in B-cell homeostasis and the generation of a peripheral demand for B cells had stimulated enhanced B lymphopoiesis in the BM of the old mice. Indeed, analysis of the BM of these mice revealed that the senescent B lymphopoiesis had in fact been reactivated. Inventors found a revival of B lymphopoiesis in the BM of old mice that had been subjected to multiple rounds of B-cell depletion, with profound increases in the frequencies of pro/preB and immature B cells (FIG. 6D compared to BM from an untreated old mouse, FIG. 1B). The increase in precursor frequencies was further confirmed by AA4.1 staining (FIG. 6E compared to FIG. 1H). These frequencies of precursor B cells were similar to those in the BM of young mice (FIGS. 1A and 1G). Moreover, inventors found that splenic B cells bearing the old-like phenotype PanCD45+/B220^{lo} were eliminated in the treated mice and replaced with a rejuvenated population of B cells expressing a young-like phenotype (FIG. 6F compared to FIG. 1N). These observations indicated that the potential to produce a high output of B lymphocytes is fully retained in the aged BM and that the rate of B lymphopoiesis is determined by homeostatic demands.

Inventors next tested whether B-cell depletion also rejuvenates the peripheral repertoire. For this, inventors applied the B-cell depletion strategy to the repertoire-reporting 3-83 Tg mouse model. Similar to the old wt mice, inventors found that B-cell depletion revived B lymphopoiesis in the old 3-83 Tg mice, as revealed by a more than 15-fold increase in the frequency of B220+/ID+/AA4.1+ cells in the BM (FIGS. 6G-J). Analysis of the peripheral repertoire revealed that the old-like repertoire had been replaced following B-cell depletion with a young-like one, in which most B cells expressed the transgenic receptor (FIGS. 6K-N). Inventors thus concluded that B-cell homeostasis, which is set by the longevity of the peripheral B cells, is a primary mechanism for senescence of the B lineage. The alteration of this homeostasis in aging revives B lymphopoiesis in the BM and rejuvenates the peripheral repertoire.

### Example 5

### Rejuvenated B Lineage in Old Mice Confers an Enhanced Immune Responsiveness

One of the most important changes in aging is the failure to mount protective antibody responses to vaccination and to infectious agents, due to a reduction in the diversity of the peripheral repertoire. Because the peripheral repertoire of the aged mice was replaced and rejuvenated after B-cell depletion (see Example 4, above), inventors next tested whether this new repertoire conferred an increased competence to mount an antibody response against a new antigenic challenge. As shown in FIG. 7, old wt mice produced a very poor anti-NP IgG1 response compared with young mice, and detectable antibodies were found in only one third of the old immunized mice. In sharp contrast, a significant (p=0.03) 4-5-fold increase in anti-NP IgG1 antibody titers was found in old wt mice that were subjected to only one round of B-cell depletion, and detectable amounts of antibodies were found in all treated mice (FIG. 7). Hence, the reconstituted B-cell repertoire in old mice that were subjected to one round of B-cell depletion restored in part the capacity to mount an antibody response to a new antigenic challenge.

### Discussion

Taken together, the present inventors have shown that homeostatic pressures that are set by peripheral long-lived B cells regulate the senescence of B lymphopoiesis. When the long-lived B cells do not accumulate, as in the CD19−/− and Ii−/− mice, or are eliminated, as in the BAFF-R^{f/f} Mx-cre mice, B lymphopoiesis does not become senescent.

Moreover, the present results indicated that senescence in the B lineage can be reversed. The entrance into senescence, as well as the revival of the B lineage in aging, is largely regulated by homeostatic demands and crosstalk between the B cells in the periphery and the BM. However, the restoration of B lymphopoiesis in the aged population may not occur rapidly and a long-term deficiency or multiple depletions may be necessary to measurably accomplish it.

Restoring the immune competence to mount antibody responses to new antigens is of major importance to the elderly population. As shown by the present results, mice treated for one round of B cell depletion developed a significant increased antibody response to NP-CGG challenge. The interpretation of these findings is that the young-like peripheral repertoire that is reconstructed in old mice after B cell depletion is more competent in recognition and responsiveness to new antigenic challenge. Thus, the replacement of the peripheral B cell pool with a young B cell pool may be used as a tool to restore immune competence and efficacy of vaccination in aging.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-17. (canceled)

18. A method of altering peripheral B cell populations in a healthy subject, the method comprising, administering to the subject a therapeutically effective amount of an agent capable of depleting peripheral B cells, rejuvenating the peripheral repertoire and restoring B cell competence, thereby altering peripheral B cell populations in the healthy subject.

19. A method of treating an aged-immune compromised subject, the method comprising administering to the subject a therapeutically effective amount of an agent capable of depleting peripheral B cells, rejuvenating the peripheral repertoire and restoring B cell competence thereby treating the aged-immune compromised subject.

20. A method of treating an infectious disease in a subject in need thereof, the method comprising administering to the subject a therapeutically effective amount of an agent capable of depleting peripheral B cells, rejuvenating the peripheral repertoire and restoring B cell competence, thereby treating the infectious disease in the subject.

21. The method of claim 18, wherein said therapeutically effective amount is sufficient to allow generation of new B cells in the bone marrow of the subject.

22. The method of claim 19, wherein said therapeutically effective amount is sufficient to allow generation of new B cells in the bone marrow of the subject.

23. The method of claim 20, wherein said therapeutically effective amount is sufficient to allow generation of new B cells in the bone marrow of the subject.

24. The method of claim 19, wherein the subject is at least about 40 years old.

25. The method of claim 18, wherein said agent comprises a targeting moiety.

26. The method of claim 18, wherein said agent comprises a targeting moiety.

27. The method of claim 18, wherein said agent comprises a targeting moiety.

28. The method of claim 25, wherein said targeting moiety comprises an antibody.

29. The method of claim 26, wherein said targeting moiety comprises an antibody.

30. The method of claim 27, wherein said targeting moiety comprises an antibody.

31. The method of claim 28, wherein said antibody comprises an anti-B cell antibody.

32. The method of claim 29, wherein said antibody comprises an anti-B cell antibody.

33. The method of claim 30, wherein said antibody comprises an anti-B cell antibody.

34. The method of claim 31, wherein said anti-B cell antibody is selected from the group consisting of an anti-CD20 antibody, an anti-CD22 antibody and an anti-CD19 antibody.

35. The method of claim 32, wherein said anti-B cell antibody is selected from the group consisting of an anti-CD20 antibody, an anti-CD22 antibody and an anti-CD19 antibody.

36. The method of claim 33, wherein said anti-B cell antibody is selected from the group consisting of an anti-CD20 antibody, an anti-CD22 antibody and an anti-CD19 antibody.

37. The method of claim 28, wherein said antibody comprises an antibody targeting a B cell survival factor.

38. The method of claim 29, wherein said antibody comprises an antibody targeting a B cell survival factor.

39. The method of claim 30, wherein said antibody comprises an antibody targeting a B cell survival factor.

40. The method of claim 37, wherein said antibody targeting a B cell survival factor comprises an anti-Blys (BAFF) antibody.

41. The method of claim 38, wherein said antibody targeting a B cell survival factor comprises an anti-Blys (BAFF) antibody.

42. The method of claim 39, wherein said antibody targeting a B cell survival factor comprises an anti-Blys (BAFF) antibody.


SEQUENCE LISTING (1 sequence)

SEQ ID NO: 1 - DNA, 19 bp, Artificial sequence
  CD20 siRNA target sequence
ccactcttcaggaggatgt